(19) United States
(12) Patent Application Publication
(10) Pub. No.: US 2025/0272496 A1
(43) Pub. Date: Aug. 28, 2025

(54) SENTENCE GENERATION DEVICE, SENTENCE GENERATION METHOD, AND RECORDING MEDIUM

(71) Applicant: NEC Corporation, Tokyo (JP)

(72) Inventors: Akira Yamauchi, Tokyo (JP); Mayu Kaya, Tokyo (JP); Yasunari Takei, Tokyo (JP); Junya Ushikoshi, Tokyo (JP); Junichi Yahara, Tokyo (JP); Daisuke Fukai, Tokyo (JP); Ryosuke Ito, Tokyo (JP); Ryosuke Matsumura, Tokyo (JP)

(73) Assignee: NEC Corporation, Tokyo (JP)

(21) Appl. No.: 19/010,324

(22) Filed: Jan. 6, 2025

(30) Foreign Application Priority Data
Feb. 28, 2024 (JP) 2024-029013

(51) Int. Cl. G06F 40/30; G16H 10/60

(52) U.S. Cl. CPC G06F 40/30; G16H 10/60

(57) ABSTRACT

A sentence generation device includes at least one memory configured to store instructions; and at least one processor configured to execute the instructions to: display, for each type of medical sentences, icons indicating items to be included in a medical sentence; receive designation of any of icons of items for each of which a summary sentence is generated; input an instruction sentence related to the designated icon into a language model to generate the summary sentence based on electronic medical record information; and output the generated summary sentence.

This application is based upon and claims the benefit of priority from Japanese Patent Application No. 2024-029013, filed on Feb. 28, 2024, the disclosure of which is incorporated herein in its entirety by reference.

### TECHNICAL FIELD

The present disclosure relates to a sentence generation device, a sentence generation method, and a recording medium.

### BACKGROUND ART

There is a technique for reducing a burden of creating a document created by a doctor or the like.

For example, Reference Document 1 (JP 2015-138402 A) discloses extracting a written expression described in medical information, interpreting the extracted written expression as a medical fact, extracting a confirmed disease that is a disease diagnosed confirmedly from the medical information, and creating an explanation of a diagnosis basis in the confirmed disease diagnosed confirmedly based on the medical fact.

### SUMMARY

An object of the present disclosure is to provide a sentence generation device and the like capable of easily creating a summary sentence of a medical record of a patient.

A sentence generation device according to an aspect of the present disclosure includes at least one memory configured to store instructions; and at least one processor configured to execute the instructions to: display, for each type of medical sentences, icons indicating items to be included in a medical sentence; receive designation of any of icons of items for each of which a summary sentence is generated; input an instruction sentence related to the designated icon into a language model to generate the summary sentence based on electronic medical record information; and output the generated summary sentence.

In a sentence generation method according to an aspect of the present disclosure executed by a computer, the method includes displaying, for each type of medical sentences, icons indicating items to be included in a medical sentence, receiving designation of any of icons of items for each of which a summary sentence is generated, inputting an instruction sentence related to the designated icon into a language model to generate the summary sentence based on electronic medical record information, and outputting the generated summary sentence.

A program according to an aspect of the present disclosure causes a computer to execute displaying, for each type of medical sentences, icons indicating items to be included in a medical sentence, receiving designation of any of icons of items for each of which a summary sentence is generated, inputting an instruction sentence related to the designated icon into a language model to generate the summary sentence based on electronic medical record information, and outputting the generated summary sentence.

The program may be stored in a non-transitory computer-readable recording medium.

### BRIEF DESCRIPTION OF THE DRAWINGS

Exemplary features and advantages of the present disclosure will become apparent from the following detailed description when taken with the accompanying drawings in which:

FIG. 1 is a block diagram illustrating a configuration of a sentence generation device according to the present disclosure;

FIG. 2 is a diagram illustrating a hardware configuration in which the sentence generation device according to the present disclosure is implemented by a computer device and its peripheral device;

FIG. 3 is a diagram illustrating an example of a generation screen of the summary sentence according to the present disclosure;

FIG. 4 is a diagram illustrating an example of a generation screen of the summary sentence according to the present disclosure; and

FIG. 5 is a flowchart illustrating an operation of the sentence generation according to the present disclosure.

### EXAMPLE EMBODIMENT

Hereinafter, example embodiments of a sentence generation device, a sentence generation method, a program, and a non-transitory recording medium recording the program according to the present disclosure will be described in detail with reference to the drawings. The present example embodiment does not limit the disclosed technology.

### First Example Embodiment

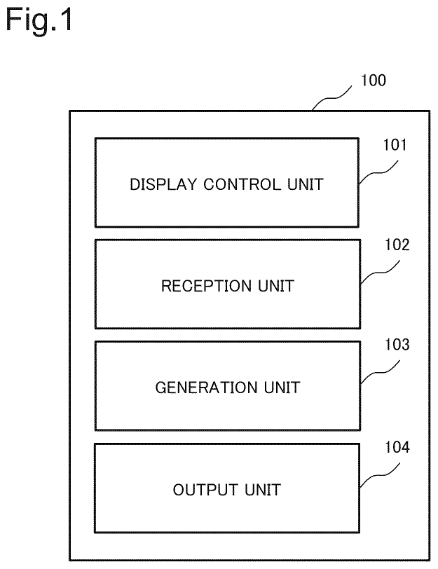

FIG. 1 is a block diagram illustrating a configuration of a sentence generation device 100 according to the present disclosure. As illustrated in FIG. 1, the sentence generation device 100 includes a display control unit 101, a reception unit 102, a generation unit 103, and an output unit 104.

The sentence generation device 100 is a device that generates a medical sentence in which medical records and the like of a patient are collected. An example of the medical sentence includes a medical summary describing a diagnosis name, a symptom, a finding, a treatment content, a nursing content, a treatment course, and the like in a summarized manner. The summary includes a discharge summary created at the timing of transfer or discharge, a weekly summary, a monthly summary, and the like created for each predetermined period. However, the medical sentence of the present disclosure is not limited to these sentences as long as it is a medical sentence in which a medical record or the like of a patient is collected.

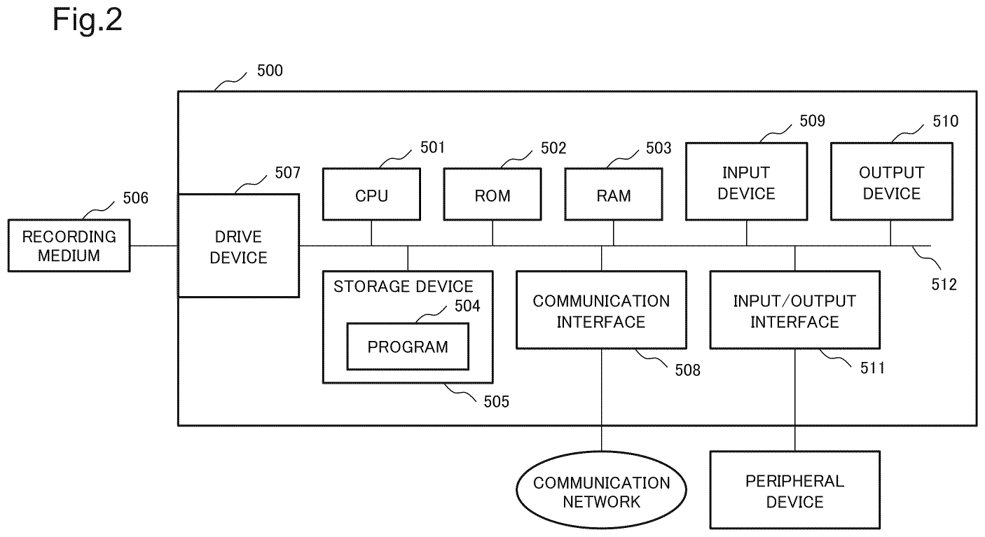

FIG. 2 is a diagram illustrating an example of a hardware configuration in which the sentence generation device 100 according to the present disclosure is achieved by a computer device 500 including a processor. As illustrated in FIG. 2, the sentence generation device 100 includes a central processing unit (CPU) 501, a memory such as, a read only memory (ROM) 502 and a random access memory (RAM) 503, a storage device 505 such as a hard disk that stores a program 504, a communication interface (I/F) 508 for network connection, and an input/output interface 511 that inputs and outputs data.

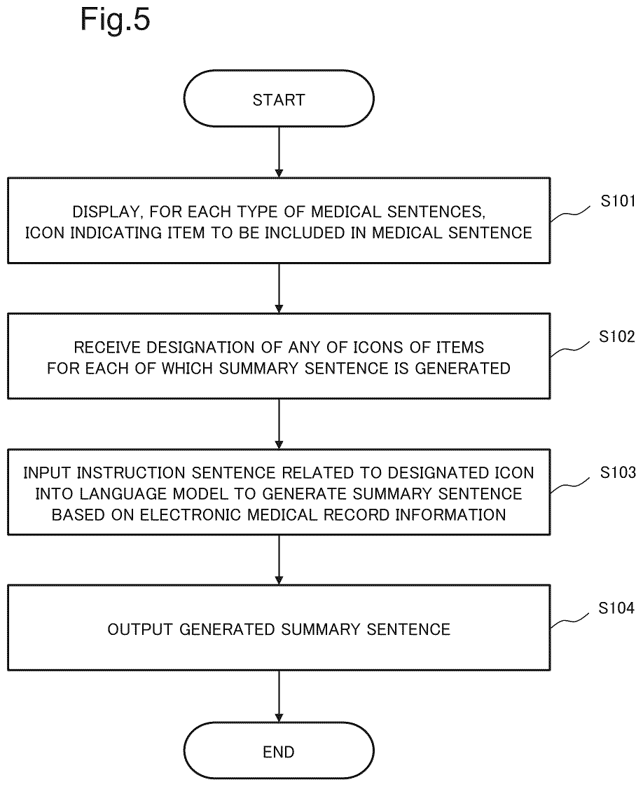

The CPU 501 operates the operating system to control the entire sentence generation device 100 according to the present disclosure. The CPU 501 reads a program and data from a recording medium 506 attached to a drive device 507 or the like to a memory, for example. The CPU 501 functions as the display control unit 101, the reception unit 102, the generation unit 103, the output unit 104, and part thereof in the present disclosure, and executes processing or a command in the flowchart illustrated in FIG. 5 to be described later based on a program.

The recording medium 506 is, for example, an optical disk, a flexible disk, a magnetic optical disk, an external hard disk, a semiconductor memory, or the like. The recording medium as part of the storage device is a nonvolatile storage device, and records a program therein. The program may be downloaded from an external computer (not illustrated) connected to a communication network.

An input device 509 is achieved by, for example, a mouse, a keyboard, a built-in key button, and the like, and is used for an input operation. The input device 509 is not limited to a mouse, a keyboard, and a built-in key button, and may be, for example, a touch panel. An output device 510 is achieved by, for example, a display, and is used to check an output.

As described above, the sentence generation device 100 illustrated in FIG. 1 is implemented by the computer hardware illustrated in FIG. 2. However, the means for achieving each unit included in the sentence generation device 100 in FIG. 1 is not limited to the above-described configuration. The sentence generation device 100 may be achieved by one physically coupled device, or may be achieved by a plurality of devices by connecting two or more physically separated devices in a wired or wireless manner. For example, the input device 509 and the output device 510 may be connected to the computer device 500 via a network. The sentence generation device 100 illustrated in FIG. 1 can also be configured by cloud computing or the like.

The display control unit 101 is a means that displays, for each type of medical sentences, icons indicating items to be included in a medical sentence. For example, the display control unit 101 displays an icon on a display screen on which an application program for displaying a medical sentence is displayed. The item is a title when classified according to the content described in the medical sentence, and is, for example, basic information about a patient, a surgical history, a hospitalization history, sickness name information, a prescription history, or the like. Each of the icons is associated with an instruction sentence to be input into the language model. The instruction sentence includes an instruction for acquiring electronic medical record information necessary for creating a summary sentence of each item and generating a summary of the acquired electronic medical record information. The instruction sentence may include giving a specific role such as an internal medicine doctor or a surgical doctor into the language model. In this case, a summary sentence related to the given role can be generated. The instruction sentence is, for example, a prompt used as input of a large-scale language model, but is not limited to the prompt. An example of a prompt of the present disclosure includes “You are an internal medicine doctor. Please create a hospitalization record of patient *** in this month. The admission record should include the chief complaint and course of treatment.” and the like.

The prompt may include generating the summary sentence to satisfy a predetermined generation condition. Examples of the generation condition include a condition for the content or a format to be included in the summary sentence, for example, a condition of not writing the content of presumption, a condition of using a gentle wording of a desu/masu style, a condition of describing in itemized form, and the like. In a case where the summary is required to be created within predetermined days after discharge such as a discharge summary, the reception unit 102 may receive setting of a generation deadline of the summary sentence.

The type of the medical sentence varies depending on the purpose of creation, the creator, and who the sentence is addressed to. For example, the nursing summary is a sentence created for a medical professional at a receiving destination in such a way that nursing can be smoothly continued at the next receiving destination when a patient transfers or leaves a hospital or when a hospital ward is changed, and is created by a nurse. In addition, the types of medical sentences include a sentence created by a doctor to a medical professional and a sentence created by a doctor or a nurse to a patient.

The reception unit 102 is a means that receives designation of any of icons of items for each of which a summary sentence is generated. The reception unit 102 receives designation of an icon by an operation on the screen by the user on the screen of the application program for generating the summary sentence. The number of icons received by the reception unit 102 may be singular or plural. The reception unit 102 may receive addition of an icon by an operation on the screen by the user. The added icon is also associated with the instruction sentence related to the item.

The generation unit 103 is a means that inputs an instruction sentence related to the received icon into the language model to generate a summary sentence based on the electronic medical record information. The generation unit 103 acquires text information related to the item indicated by the icon from the electronic medical record information and summarizes the acquired electronic medical record information to generate a summary sentence using the language model. For example, the generation unit 103 may input an instruction sentence associated with an icon into a language model stored in the storage device 505, or may input an instruction sentence associated with an icon into a language model stored in an external system. When a generation deadline is set, the generation unit 103 generates a summary sentence in time for the deadline. The generation unit 103 may generate the summary sentence using the nighttime during which normal work is not performed.

For example, the language model performs processing related to a prompt related to the received icon to output a summary sentence. The language model may be a large-scale language model learned with a large amount of text data by machine learning or a learned model obtained by transfer learning the large-scale language model. Examples of the large-scale language model can include generative pre-training-2 (GPT-2), GPT-3, or GPT-4. Examples of the large-scale language model may include text-to-text transfer transformer (T5), bidirectional encoder representations from transformers (BERT), robustly optimized BERT approach (ROBERTa), and efficiently learning an encoder that classifies token replacements accurately (ELECTRA).

The generation unit 103 executes processing of, for example, generating a summary sentence in the following procedure using the language model. That is, the generation unit 103 extracts the electronic medical record information about the patient necessary for generating the summary sentence from the server device or the like of the medical institution, and gives the electronic medical record information together with the prompt into the language model to generate the summary sentence. The generation unit 103 may give a patient ID or the like as a prompt to the language model and allow the language model to refer to the electronic medical record information about the patient and generate a summary sentence.

The language model may generate a summary sentence using a template sentence prepared in advance. The template sentence is prepared for each type of medical sentences, for example, and is stored in the storage device 505, for example. The generation unit 103 generates a summary sentence by giving the template sentence and the electronic medical record information to the language model. The template sentence is data including a text in which patient's unique information is excluded from the summary sentence. The unique information is information unique to each patient, and includes, for example, personal information such as a name, a date of birth, and electronic medical record information about the patient. By generating a summary sentence using the template sentence, it is possible to generate a summary sentence in a format unified by a doctor or a medical institution.

The output unit 104 is a means that outputs the generated summary sentence on a display device such as a display. The output unit 104 outputs the summary sentence on a screen of an application program for creating the summary sentence, for example. The output unit 104 may transmit the generated summary sentence to a terminal device used by the user, or may output an operation performed by the user to generate the summary sentence of the target patient as a trigger.

The output unit 104 may display an annotation for a terminology included in the summary sentence. The terminology may, for example, be stored in the storage device 505 with an annotation of the terminology, and the output unit 104 may collate with the terminology stored in the storage device 505 to display the annotation associated with the terminology. As a result, even in a case where a summary sentence is generated for a medical institution staff, other than doctors, who does not have knowledge about the terminology, the recipient can understand the content of the summary sentence.

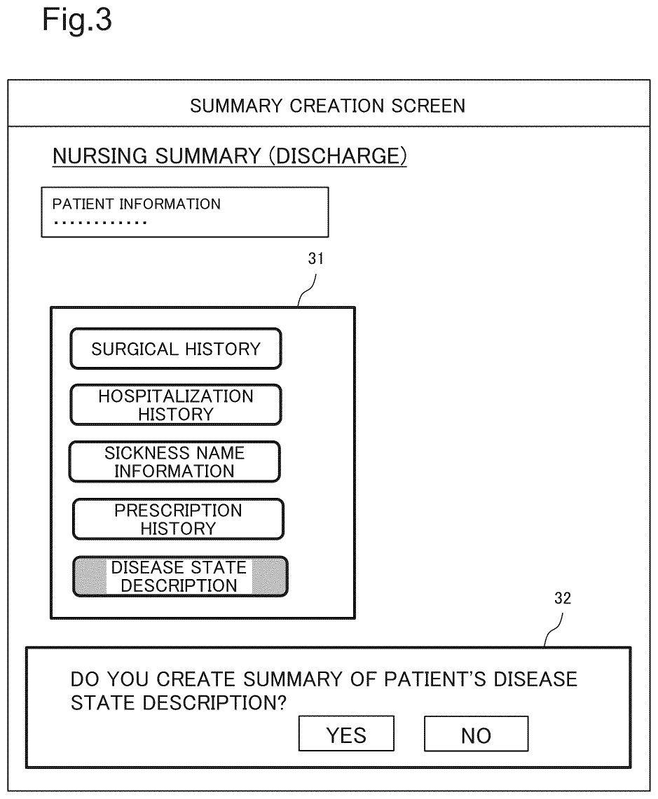

FIG. 3 is an example of a generation screen of the summary sentence according to the present disclosure. The example of FIG. 3 is a creation screen of the nursing summary created by a nurse. As illustrated in FIG. 3, the screen includes an icon 31 indicating items to be included in the medical sentence and a display 32 including a button for selecting whether to generate the summary sentence of the patient. In the example of FIG. 3, the reception unit 102 receives designation of any of the icons 31 of the items for each of which the summary sentence is generated on the screen for generating the summary sentence. As illustrated in FIG. 3, the designated icon 31 may be highlighted by bolding or changing the color.

The output unit 104 may display electronic medical record information that is a basis of the information included in the summary sentence. The information included in the summary sentence includes a symptom course, a test result, a treatment course, information about a prescribed medicine, findings of the patient by a doctor, and the like of the patient, and is electronic medical record information used when the generation unit 103 generates the summary sentence. For example, the output unit 104 may display a link of the electronic medical record information of the citation source included in the summary sentence to output the electronic medical record information. The output unit 104 may output information identifying the electronic medical record information of the citation source. The information identifying the electronic medical record information is, for example, information such as an input date and time of the electronic medical record information or a person who input the information.

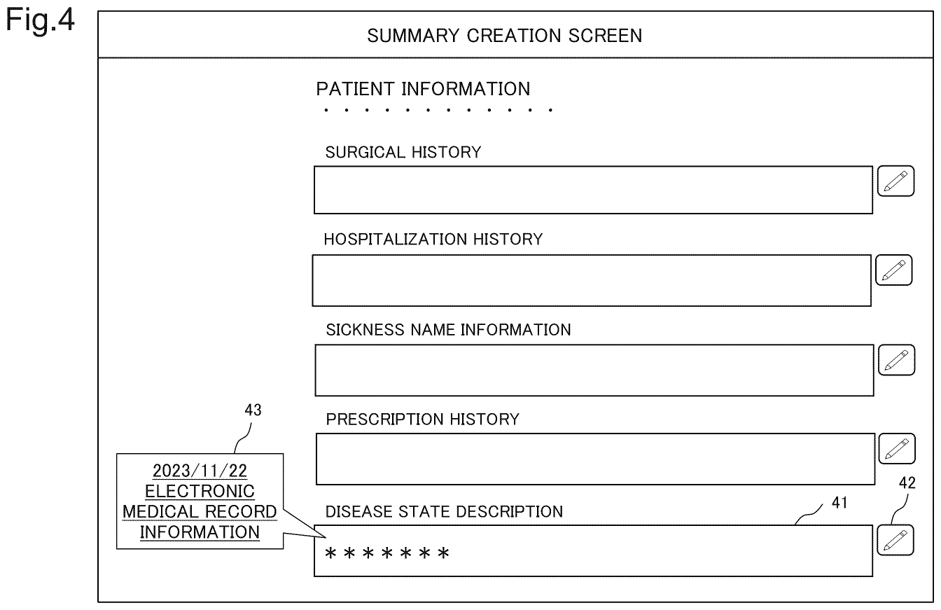

FIG. 4 is an example of a generation screen of the summary sentence according to the present disclosure. In the example of FIG. 4, a screen on which a summary sentence 41 is displayed for each item is illustrated, and a summary sentence of a “disease state description” of the received item is displayed. In the example of FIG. 4, when an icon 42 is selected, the summary sentence can be edited by the user on the screen. In the example of FIG. 4, a basis 43 of the electronic medical record information is displayed. The output example of the summary sentence in FIG. 4 is an example of the present disclosure, and the summary sentence may be output by other methods.

The operation of the sentence generation device 100 configured as described above will be described with reference to the flowchart of FIG. 5.

FIG. 5 is a flowchart illustrating an outline of the operation of the sentence generation device 100 according to the present disclosure. The processing according to this flowchart may be executed based on program control by the processor described above. The processing according to this flowchart is started as a trigger when the user performs an operation for creating a summary sentence.

As illustrated in FIG. 5, first, the display control unit 101 displays, for each type of medical sentences, icons indicating items to be included in a medical sentence (step S101). Next, the reception unit 102 receives designation of any of the icons of the items for each of which the summary sentence is generated (step S102). The generation unit 103 inputs an instruction sentence related to the designated icon into the language model to generate a summary sentence based on the electronic medical record information (step S103). Finally, the output unit 104 outputs the generated summary sentence (step S104). The sentence generation device 100 thus ends the sentence generation operation.

In the sentence generation device 100, the generation unit 103 inputs an instruction sentence related to the designated icon into the language model to generate a summary sentence based on the electronic medical record information. As a result, a medical professional who is a user can generate a summary sentence by performing an operation for generating the summary sentence. Therefore, the summary sentence of the medical record of the patient can be easily created.

Each modification of the above-described first example embodiment will be described. In the present disclosure, each modification can be implemented by an any combination.

### First Modification of First Example Embodiment

In the first example embodiment, the sentence generation device 100 may generate a summary sentence using a sample sentence of a summary sentence prepared in advance. The sample sentence is a sample of a summary sentence of the same item used in the past, and is prepared for each doctor or each clinical department and stored in the storage device 505, for example. The reception unit 102 receives an instruction including generation of a summary sentence using a sample sentence as a generation condition. The generation unit 103 inputs the instruction sentence and the sample sentence into the language model to generate a summary sentence. By generating the summary sentence using the sample sentence, it is possible to generate a summary sentence in a format familiar to a doctor or the like who is a user.

### Second Modification of First Example Embodiment

The reception unit 102 may further receive whether to use the output summary sentence. For example, the reception unit 102 receives an answer as to whether the user uses the output sentence on the screen on which the summary sentence is output. When receiving that the output summary sentence is not used, the reception unit 102 may further receive that the summary sentence is generated by changing the generation condition. In this case, the reception unit 102 may receive at least one of a sentence length, a degree of summarization, and electronic medical record information included in the summary sentence as a condition for regenerating the summary sentence. The length of the sentence is indicated by, for example, the number of characters and the number of lines. The summarization degree indicates, for example, how much the sentence is summarized with respect to the length of the original sentence. When receiving the regeneration of the summary sentence, the reception unit 102 updates the prompt by including the received generation condition in the prompt. The generation unit 103 inputs the updated prompt into the language model to regenerate the summary sentence.

In the present modification, the reception unit 102 receives whether to use the output summary sentence. This allows the user to confirm the content of the summary sentence. When receiving that the output summary sentence is not used, the reception unit 102 receives that the summary sentence is generated by changing the generation condition. As a result, it is possible to regenerate the summary sentence reflecting the user's intention to correct.

### Third Modification of First Example Embodiment

In each example embodiment of the present disclosure, the generation unit 103 may generate a summary sentence using a plurality of different learned models, and the output unit 104 may output each generated summary sentence. In this case, the reception unit 102 may receive whether to use the summary sentence generated by which model. Examples of the plurality of learned models can include a large-scale language model disclosed as open source and a language model using a unique engine. In this case, the user can select the better summary sentence.

A medical professional such as a doctor creates a summary sentence (summary) of a medical record using electronic medical record information in order to record medical care content to a patient. However, it takes time to search for necessary sentences from the electronic medical record information or to summarize the sentences in order to create a summary sentence of a medical record. The quality and impression of the summary sentence vary depending on the creator.

According to an example of an effect of the present disclosure, a summary sentence of a medical record of a patient can be easily created.

While the present disclosure is described with reference to example embodiments thereof, the present disclosure is not limited to these example embodiments. Various modifications that can be understood by those of ordinary skill in the art in the art can be made to the configuration and details of the present disclosure within the scope of the present disclosure.

For example, although the plurality of operations is described in order in the form of a flowchart, the order of description does not limit the order in which the plurality of operations is executed. Therefore, when each example embodiment is implemented, the order of the plurality of operations can be changed within a range that does not interfere with the content.

Further, it is noted that the inventor's intent is to retain all equivalents of the claimed disclosure even if the claims are amended during prosecution.

Some or all of the above example embodiments may be described as the following Supplementary Notes, but are not limited to the following.

### (Supplementary Note 1)

A sentence generation device including
a display control means for displaying, for each type of medical sentences, icons indicating items to be included in the medical sentence,
a reception means for receiving designation of any of icons of items for each of which a summary sentence is generated,
a generation means for inputting an instruction sentence related to the designated icon into a language model to generate the summary sentence based on electronic medical record information, and
an output means for outputting the generated summary sentence.

### (Supplementary Note 2)

The sentence generation device according to Supplementary Note 1, wherein the generation means acquires text information related to an item indicated by each icon from the electronic medical record information and summarizes the acquired electronic medical record information to generate the summary sentence using the language model.

### (Supplementary Note 3)

The sentence generation device according to Supplementary Note 1, wherein the output means displays the generated summary sentence in an editable manner.

### (Supplementary Note 4)

The sentence generation device according to Supplementary Note 1, wherein the output means displays electronic medical record information that is a basis of information included in the summary sentence.

### (Supplementary Note 5)

The sentence generation device according to Supplementary Note 1, wherein
the reception means further receives an instruction including generating the summary sentence using a template sentence prepared in advance as a generation condition, and
the generation means inputs the instruction sentence and the template sentence into the language model, and applies electronic medical record information to the template sentence to generate the summary sentence.

### (Supplementary Note 6)

The sentence generation device according to Supplementary Note 1, wherein
the reception means further receives an instruction including generating the summary sentence using a sample sentence as a generation condition, and
the generation means inputs the instruction sentence and the sample sentence into the language model to generate the summary sentence.

### (Supplementary Note 7)

The sentence generation device according to Supplementary Note 1, wherein the reception means receives whether to use the output summary sentence.

### (Supplementary Note 8)

The sentence generation device according to Supplementary Note 1, wherein
the generation means generates summary sentences using a plurality of the different language models,
the output means outputs the generated summary sentences, and
the reception means receives whether a sentence generated by which language model is used.

### (Supplementary Note 9)

A sentence generation method executed by a computer, the method including
displaying, for each type of medical sentences, icons indicating items to be included in a medical sentence,
receiving designation of any of icons of items for each of which a summary sentence is generated,
inputting an instruction sentence related to the designated icon into a language model to generate the summary sentence based on electronic medical record information, and
outputting the generated summary sentence.

### (Supplementary Note 10)

A program causing a computer to execute
displaying, for each type of medical sentences, icons indicating items to be included in a medical sentence,
receiving designation of any of icons of items for each of which a summary sentence is generated,
inputting an instruction sentence related to the designated icon into a language model to generate the summary sentence based on electronic medical record information, and
outputting the generated summary sentence.

Some or all of the configurations described in Supplementary Notes 2 to 8 dependent on the above-described Supplementary Note 1 can also be dependent on Supplementary Notes 9 and 10 by the dependency relationship similar to that of Supplementary Notes 2 to 8. Furthermore, some or all of the configurations described as the Supplementary Notes can be similarly dependent on not only the Supplementary Notes 1, 9, and 10, but also various pieces of hardware and software, and various recording devices or systems for recording software without departing from the above-described example embodiments.

### REFERENCE SIGNS LIST

100 sentence generation device
101 display control unit
102 reception unit
103 generation unit
104 output unit
500 computer device
501 CPU
502 ROM
503 RAM
504 program
505 storage device
506 recording medium
507 drive device
508 communication interface
511 input/output interface
512 bus

1. A sentence generation device comprising:
at least one memory configured to store instructions; and
at least one processor configured to execute the instructions to:
display, for each type of medical sentences, icons indicating items to be included in a medical sentence;
receive designation of any of icons of items for each of which a summary sentence is generated;
generate the summary sentence based on electronic medical record information by inputting an instruction sentence related to the designated icon into a language model; and
output the generated summary sentence.

2. The sentence generation device according to claim 1, wherein the at least one processor is further configured to execute the instructions to
acquire text information related to an item indicated by each icon from electronic medical record information and summarize the acquired electronic medical record information to generate the summary sentence using the language model.

3. The sentence generation device according to claim 1, wherein the at least one processor is further configured to execute the instructions to
display the generated summary sentence in an editable manner.

4. The sentence generation device according to claim 1, wherein the at least one processor is further configured to execute the instructions to
display electronic medical record information that is a basis of information included in the summary sentence.

5. The sentence generation device according to claim 1, wherein the at least one processor is further configured to execute the instructions to:
receive an instruction including generating the summary sentence using a template sentence prepared in advance as a generation condition; and
generate the summary sentence by inputting the instruction sentence and the template sentence into the language model and applying electronic medical record information to the template sentence.

6. The sentence generation device according to claim 1, wherein the at least one processor is further configured to execute the instructions to:
receive an instruction including generating the summary sentence using a sample sentence as a generation condition; and
generate the summary sentence by inputting the instruction sentence and the sample sentence into the language model.

7. The sentence generation device according to claim 1, wherein the at least one processor is further configured to execute the instructions to
receive whether to use the output summary sentence.

8. The sentence generation device according to claim 1, wherein the at least one processor is further configured to execute the instructions to:
generate summary sentences using a plurality of different language models;
output the generated summary sentences; and
receive whether a sentence generated by which language model is used.

9. A sentence generation method comprising:
displaying, for each type of medical sentences, icons indicating items to be included in a medical sentence;
receiving designation of any of icons of items for each of which a summary sentence is generated;
generating the summary sentence based on electronic medical record information by inputting an instruction sentence related to the designated icon into a language model; and
outputting the generated summary sentence.

10. The sentence generation method according to claim 9, further comprising
acquiring text information related to an item indicated by each icon from electronic medical record information and summarizing the acquired electronic medical record information to generate the summary sentence using the language model.

11. The sentence generation method according to claim 9, further comprising
displaying the generated summary sentence in an editable manner.

12. The sentence generation method according to claim 9, further comprising
displaying electronic medical record information that is a basis of information included in the summary sentence.

13. The sentence generation method according to claim 9, further comprising:
receiving an instruction including generating the summary sentence using a template sentence prepared in advance as a generation condition; and
generating the summary sentence by inputting the instruction sentence and the template sentence into the language model and applying electronic medical record information to the template sentence.

14. The sentence generation method according to claim 9, further comprising:
receiving an instruction including generating the summary sentence using a sample sentence as a generation condition; and
generating the summary sentence by inputting the instruction sentence and the sample sentence into the language model.

15. A non-transitory computer-readable recording medium that records a program for causing a computer to execute:
displaying, for each type of medical sentences, icons indicating items to be included in a medical sentence;
receiving designation of any of icons of items for each of which a summary sentence is generated;
generating the summary sentence based on electronic medical record information by inputting an instruction sentence related to the designated icon into a language model; and
outputting the generated summary sentence.

16. The recording medium, according to claim 15, that records the program for causing the computer to further execute
acquiring text information related to an item indicated by each icon from electronic medical record information and summarizing the acquired electronic medical record information to generate the summary sentence using the language model.

17. The recording medium, according to claim 15, that records the program for causing the computer to further execute
displaying the generated summary sentence in an editable manner.

18. The recording medium, according to claim 15, that records the program for causing the computer to further execute
displaying electronic medical record information that is a basis of information included in the summary sentence.

19. The recording medium, according to claim 15, that records the program for causing the computer to further execute:
receiving an instruction including generating the summary sentence using a template sentence prepared in advance as a generation condition; and
generating the summary sentence by inputting the instruction sentence and the template sentence into the language model and applying electronic medical record information to the template sentence.

20. The recording medium, according to claim 15, that records the program for causing the computer to further execute:
receiving an instruction including generating the summary sentence using a sample sentence as a generation condition; and
generating the summary sentence by inputting the instruction sentence and the sample sentence into the language model.
